(19) United States
(12) Patent Application Publication
(10) Pub. No.: US 2012/0277326 A1
(43) Pub. Date: Nov. 1, 2012

(54) BIOMARKERS OF CANCER

(76) Inventors: Douglas D. Taylor, Louisville, KY (US); Cicek Gercel-Taylor, Louisville, KY (US)

(21) Appl. No.: 13/510,849

(22) PCT Filed: Nov. 19, 2010
(86) PCT No.: PCT/US10/57427

§ 371 Date: Jul. 18, 2012

Related U.S. Application Data
(60) Provisional application No. 61/263,325, filed on Nov. 20, 2009.

(51) Int. Cl. C40B 30/04; G01N 33/573; G01N 21/76; G01N 33/574; A61K 35/00

(52) U.S. Cl. 514/789; 436/501; 506/9; 435/7.4

(57) ABSTRACT

Methods for diagnosis and staging of ovarian cancer, based on relative immunoreactivity of different IgG subclasses of autoantibodies, autoantibodies to defined antigens, e.g., antigens with specific subcellular localization, are described.

### CLAIM OF PRIORITY

This application claims the benefit of U.S. Provisional Patent Application Ser. No. 61/263,235, filed on Nov. 20, 2009, the entire contents of which are hereby incorporated by reference.

### TECHNICAL FIELD

This invention relates to biomarkers of cancer, e.g., pancreatic, lung, breast, colon, or ovarian cancer, based on relative immunoreactivity of different IgG subclasses of autoantibodies, autoantibodies to defined antigens, e.g., antigens with specific subcellular localization.

### BACKGROUND

While ovarian cancer accounts for only one third of gynecologic cancers, it results in 55% of deaths from gynecologic malignancies and 6% of all cancer deaths in women (Memarzadeh S, Berek J S., J Reprod Medicine 2001, 46:621-629; Hoskins W J. J Cell Biochem 1995; 23 (suppl):189-199). Long-term survival has not changed significantly in the last three decades, largely due to inadequate diagnostic approaches that only detect well-established cancers. Only 19% of ovarian cancers are diagnosed at Stage I (Hoskins W J., J Cell Biochem 1995, 23 (suppl):189-199), while other cancers associated with women are primarily diagnosed at Stage I (77% of endometrial cancers, 55% of breast cancers and 83% of cervical cancers). Since Stage I ovarian cancer can be cured in 90% of cases, but five-year survival for advanced disease (Stage III and IV) is less than 21%, prospects for significant improvement in survival reside in early diagnosis of disease. Current diagnostic approaches exhibit several deficiencies (Clark-Pearson D L., N Engl J Med 2009, 361:170-177). First, most biomarkers lack cancer specificity. Second, most biomarkers lack positive predictive value for early stage disease. Third, most biomarkers are unstable in the peripheral circulation. Further, the concepts of the early detection of cancer and the specific detection of early stage cancer are generally not distinguished. The identification of late stage cancer prior to symptoms will not likely impact outcome; however, detection of cancers at early stage will greatly improve survival.

While intended as a disease monitor (defining therapeutic responses, disease recurrence and progression) (Nossov V, Amneus M, Su F, Lang J, Janco J M T, Reddy S T, Farias-Eisner R., Am J Obstet Gynecol 2008, 199: 215-223), the assessment of circulating CA125 has been used to diagnose ovarian cancer (Bast R C, Badgwell D, Lu Z, Marquez R, Rosen D, Liu J, Baggerly K A, Atkinson E N, Skates S, Zhang Z, Lokshins A, Menon U, Jacobs I, Lu K., Int J Gynecol Cancer 2005, 15 (suppl 3): 274-281). CA125 is neither sensitive nor specific for de novo ovarian cancer detection, since it is elevated in less than 50% of women with stage I disease. CA125 has poor specificity, which is shown by its elevation in benign and malignant breast and colon disease, peritoneal irritants, and benign gynecologic diseases, among others (Bast R C, Badgwell D, Lu Z, Marquez R, Rosen D, Liu J, Baggerly K A, Atkinson E N, Skates S, Zhang Z, Lokshins A, Menon U, Jacobs I, Lu K., Int J Gynecol Cancer 2005, 15 (suppl 3): 274-281). Due to CA125's limited expression in early stage ovarian cancers and its association with nonmalignant pathologies, CA125, at best, exhibits a positive predictive value of 57% (Nossov V, Amneus M, Su F, Lang J, Janco J M T, Reddy S T, Farias-Eisner R., Am J Obstet Gynecol 2008, 199: 215-223).

Significant effort has been expended to identify potential markers that might substitute or complement CA125 in disease management or ultimately in screening strategies (Jacobs I J, Menon U., Mol Cell Proteomics, 3:355-366, 2004). Surface-enhanced laser desorption/ionization time-of-flight mass spectrometry (SELDI-TOF-MS) has received much attention for its use in resolving proteins in biological specimens. SELDI-TOF-MS profiling has been successfully used to differentiate ovarian, breast, prostate, and liver cancers from healthy controls (Zhang H, Kong B, Qu X, Jia L, Deng B, Yang Q., Gynecol Oncol, 102:61-66, 2006). SELDI-TOF-MS profiling of serum was significantly better than the current standard serum biomarker CA125 at distinguishing patients with ovarian cancer from those with benign ovarian disease and from healthy controls (Petricoin E F, Ardekani A M, Hitt B A, Levine P J, Fusaro V A, Steinberg S M., Lancet, 359:572-577, 2002). While these initial studies on SELDI-TOF-MS profiling are promising, translating this approach into a routine diagnostic test remains difficult. A drawback of MS techniques is that some proteins of importance may be masked by more abundant proteins in the MS as well as in the analysis of the spectrometric output. The greatest challenge in current MS approaches is the dynamic range rather than sensitivity. While removal of prevalent proteins or peptides can greatly increase the informational content acquired from particular samples, prevalent proteins such as albumin can function as carriers of protein subsets of diagnostic significance (Petricoin E F, Belluco C, Arauio R P, Liotta L A., Nature Rev Cancer 2006, 6:961-967).

Complicating the lack of specificity of current diagnostic methods is the poor stability of many biomarkers within the peripheral circulation. For antigen-based assays, such as CA125 ELISA and even SELDI-TOF-MS, once the target antigens are released from the tumor, they must saturate the immunologic antigen-processing capacity, intravasate, and reach a detectable steady-state concentration in the circulation. As a result, circulating biomarker concentrations are influenced by multiple variables, such as marker intravasation, clearance rates and protein half-lives in the blood. While stabilities of all biomarkers have not been investigated, studies on circulating p53 indicate a half-life of several hours (Angelopoulou K, Yu H, Bharaj B, Giai M, Diamandis E P., Clin Biochem 2000, 33: 53-62) and the half-life of circulating S100B protein (in melanoma) has been estimated to be only 30 minutes (Harpio R, Einarsson R., Clin Biochem 2004, 37: 512-518). In prostate cancer, total PSA and free PSA exhibit a rapid exponential degradation phase with a half-life of 4.27 and 2.14 hours, respectively (Gregorakis A K, Stefanakis S, Malovrouvas D, Petraki K, Gourgiotis D, Scorilas A., Prostate 2008, 68:759-765). In patients with intrathoracic tumors, the average half-lives of CEA, SCC, TPA and CYFRA were 36 hours, 2.2 hours, 2.5 hours and 1.5 hours, respectively (Yoshimasu T, Maebeya S, SuzumaT, Bessho T, Tanino H, Arimoto J, Sakurai T, Naito Y., Int J Biol Markers 1999, 14:99-105). In addition to short half-lives, some serum biomarkers for ovarian cancer have also been demonstrated to be highly sensitive to confounding factors, including psychological stress, time of blood draw, and uncontrolled differences in sample manipulation (Thorpe J D, Duan X, Forrest R., PLoS ONE 2007, 2: e1281). Based on mathematical models correlating biomarker detection limits with actual tumor burden, the calculated minimum tumor size leading to a positive test result was 116.7 mm3 using CA125 and ovarian cancer (Lutz A M, Willmann J K, Cochran F V, Ray P, Gambhir S S., PLoS Medicine 2008, 5:1287-1297). Since this model assumed uniform antigen production by all tumor cells, that 10% of the secreted biomarker (based on in vitro studies) reached the circulation, and that CA125 was not cleared from the blood or degraded, this calculated minimum size may be significantly underestimated.

In contrast, antibody responses are promising clinical biomarkers, since antibodies have long half-lives, are easily measured, and are stable in the peripheral circulation. Aberrant expression of cancer-associated proteins can result in autoantibody induction (Draghici S, Chatterjee M, Tainsky M A., Expert Rev Mol Diagn 2005, 5: 735-743; Gagnon A, Kim J H, Schorge J O, Ye B, Liu B, Hasselblatt K, Welch W R, Bandera C A, Mok S C., Clin Cancer Res 2008, 14: 764-771; Gercel-Taylor C, Bazzett L B, Taylor D D., Gynecol Oncol 2001, 81:71-76). In experimental animal models, circulating tumor-reactive IgG can be demonstrated soon after initial tumor development and well in advance of palpable tumor or circulating tumor antigens (Taylor D D, Gercel-Taylor C., Oncol Rep 1998 November-December, 5(6):1519-24; Nesterova M, Johnson N, Cheadle C, Cho-Chung Y S., Biochim Biophys Acta 2006, 1762: 398-403). In colorectal cancer, when comparing patients with colorectal polyps and varying stages and grades of colorectal cancer, autoantibodies against p53 appear to occur with tumor progression in the multistep colorectal carcinogenesis (Tang R, Ko M C, Wang J Y, Changchien C R, Chen H H, Chen J S, Hsu K C, Chiang J M, Hsieh L L., Int J Cancer 2001, 94:859-863). Not only are tumor reactive antibodies generated prior to detectable circulating tumor antigens, antibodies are stable and less sensitive to confounding factors relative to other serum biomarkers (Nesterova M, Johnson N, Cheadle C, Cho-Chung Y S., Biochim Biophys Acta 2006, 1762: 398-403).

### SUMMARY

As described herein, patterns of reactivity for the four IgG subclasses differ in ovarian cancer. Further, the antigenic components from different cellular compartments (membrane, nuclear or cytosol) also differ. Several of the tumor-derived antigens exhibiting shared recognition or stage-associated recognition were identified by MS to define recognition patterns of early and late stage cancers, e.g., ovarian cancer.

Thus, provided herein are methods (e.g., in vitro methods) for detecting or staging (e.g., for aiding in detecting or staging) cancer, e.g., pancreatic, lung, breast, colon, or ovarian cancer, in a subject. The methods include obtaining a sample comprising antibodies, e.g., IgG-type antibodies, from the subject; contacting the sample with one or more ovarian tumor-associated antigens, under conditions sufficient for the formation of antibody-antigen complexes; and detecting the formation of the antibody-antigen complexes, wherein the presence of complexes indicates the presence of autoantibodies against the tumor-associated antigens, and the presence of autoantibodies indicates the presence or stage of cancer, e.g., pancreatic, lung, breast, colon, or ovarian cancer, in the subject. In some embodiments, the cancer is ovarian cancer.

In some embodiments, each of the one or more tumor associated antigens is classified as either expressed in the nucleus or cytoplasm of tumor cells, e.g., pancreatic, lung, breast, colon, or ovarian tumor cells. In some embodiments, the tumor-associated antigens expressed in the nucleus are selected from the group consisting of heterogeneous nuclear ribonucleoprotein (HNRNP A2/B1), non-metastatic cells 1/non-metastatic cells 2 (NME1/NME2), zinc finger DHHC-type containing 7 isoform 2, survivin, p53, p73, nucleophosmin (B23), synovial sarcoma X common antigen or breakpoint proteins 2 and 4 (SSX2, SSX4), and homeobox A7 (HoxA7). In some embodiments, the tumor-associated antigens expressed in the cytoplasm are selected from the group consisting of pyridoxal kinase, galectin-1, heat shock protein 90, peroxiredoxin, glucose regulated protein 78, and proCathepsin D.

In some embodiments, the presence of autoantibodies that bind specifically to one or more of pyridoxal kinase, galectin-1, heat shock protein 90, zinc finger DHHC-type containing 7 isoform 2, survivin, p53, p73, glucose regulated protein 78 (GRP78) peroxiredoxin, nucleophosmin (B23), synovial sarcoma X breakpoint proteins (SSX2, SSX4), HoxA7, mucin 16, cell surface associated (Muc16), NY-ESO-1 (also known as cancer/testis antigen 1B), placental type alkaline phosphatase (PLAP), SSX common antigen, Tumor-Associated Glycoprotein 72 (TAG-72), glucose regulated protein 78 (GRP78), or CathepsinD or proCathepsin D, indicates the presence of cancer, e.g., pancreatic, lung, breast, colon, or ovarian cancer, in the subject. In some embodiments, the presence of autoantibodies that bind specifically to PLAP indicates the presence of ovarian cancer in the subject. In some embodiments, the presence of autoantibodies that bind specifically to one or more of Muc16, p53, PLAP and survivin indicates that the subject has stage III or IV ovarian cancer. In some embodiments, the presence of autoantibodies to survivin indicates the presence of lung or colon cancer in the subject.

In some embodiments, the presence of autoantibodies that bind specifically to one or more of heterogeneous nuclear ribonucleoprotein (HNRNP A2/B1) and non-metastatic cells 1/non-metastatic cells 2 (NME1/NME2) in the nucleus, and/or the presence of one or both of pyridoxal kinase, galectin-1 and heat shock protein 90 in the cytosol, indicates that the subject has stage I ovarian cancer.

In some embodiments, the presence of autoantibodies that bind specifically to one or more of zinc finger DHHC-type containing 7 isoform 2, survivin, p53, or p73 in the nucleus, and/or the presence of peroxiredoxin in the cytosol, indicates that the subject has stage III ovarian cancer.

In some embodiments, the presence of autoantibodies that bind specifically to one or more of nucleophosmin (B23), synovial sarcoma X breakpoint proteins (SSX2, SSX4), or HoxA7 in the nucleus, and/or the presence of glucose regulated protein 78 in the endoplasmic reticulum, and/or the presence of proCathepsin D in the lysosome indicates that the subject has cancer, e.g., ovarian cancer.

In some embodiments, the tumor-associated antigens are bound to a substrate, e.g., a solid surface or a bead.

In some embodiments, the tumor-associated antigens are isolated from cytoplasm of cells that are known to be cancer cells, e.g., ovarian cancer cells, or isolated from nuclei of cells that are known to be cancer cells.

In some embodiments, the methods further include communicating information regarding the presence of the autoantibodies to a health care provider or to the subject. In some embodiments, the methods further include administering a treatment (as is known in the art) for the cancer to the subject.

In another aspect, the invention provides methods (e.g., in vitro methods) of staging (e.g., for aiding in staging) ovarian cancer in a subject. The methods include obtaining a sample comprising IgG-type antibodies from the patient; contacting the sample with ovarian tumor-derived antigens, under conditions sufficient for the formation of antibody-antigen complexes; determining the subclass of the IgG antibodies bound to the antigens; and determining the relative immunoreactivity of the subclasses, wherein the relative immunoreactivity of the subclasses indicates whether the subject has early stage, middle stage, or advanced ovarian cancer.

In some embodiments, the subclasses are IgG1, IgG2, IgG3 and IgG4. In some embodiments, the presence of relative immunoreactivity of IgG2>IgG3>IgG1=IgG4 indicates a diagnosis of early stage ovarian cancer; the presence of IgG2>IgG3>IgG1>IgG4 indicates a diagnosis of middle stage ovarian cancer, and the presence of IgG2³IgG3=IgG4>IgG1 indicates advanced ovarian cancer.

In some embodiments of the methods described herein, the subject is a human, e.g., a human known to have or suspected of having ovarian cancer.

In some embodiments, the sample comprises serum from the subject.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control. Other features and advantages of the invention will be apparent from the following detailed description and figures, and from the claims.

### DESCRIPTION OF DRAWINGS

FIGS. 1A-B are representative western immunoblots of patient immunoreactivity with proteins isolated from specific cellular compartments of ovarian tumor cells: cytosol (designated as C), membrane (designated as M) and nuclear (designated as N). Recognition of proteins used patient serum diluted 1:100. Representative sera were obtained from (1A) a normal, age-matched female control and (1B) a patients with a benign ovarian mass (serous adenoma).

FIGS. 2A-C show representative western immunoblots of patient immunoreactivity with proteins isolated from specific cellular compartments of ovarian tumor cells: cytosol (designated as C), membrane (designated as M) and nuclear (designated as N). Recognition of proteins used patient serum diluted 1:100. Representative sera were obtained from (2A) a patient with Stage I ovarian cancer, (2B) with Stage II ovarian cancer and (2C) with Stage III ovarian cancer. The representative immunoblots representing IgG1, IgG2, IgG3, and IgG4 for each stage utilized the same patient's serum.

FIG. 3 is a bar graph showing the results of quantitation of western immunoblots presenting the immunoreactivity of sera from normal controls (n=20), women with benign disease (n=15) and patients with various stages of ovarian cancer (Stage I, n=15; Stage II, n=15; Stage III, n=15) against antigens derived from specific cellular compartments: cytosol (C), membrane (M) and nucleus (N). Bars represent the mean total immunoreactivity for each group of patients. The filled bars (▪) represent mean reactivity with antigens greater than 40 kD and open bars (□) represent mean reactivity with antigens less than 40 kD.

FIGS. 4A-C show 2-DIGE results of proteins recognized by Stage I and Stage III ovarian cancer patients. Cellular proteins immunopurified using affinity columns with IgG from patients with Stage I cancer were labeled with Cy2 (4A) and immunopurified using affinity columns with IgG from patients with Stage III cancer were labeled with Cy3 (4B). The gel was scanned using a Typhoon image scanner and each scan revealed one of the CyDye signals. ImageQuant software was used to generate an image presentation data including the single and overlay images (4C). In the overlay image, examples of antigens linked with stage I disease are circled in medium grey (green in original), examples of components associated with Stage III were circled in dark grey (red in original), and examples of shared components were circled in lightest grey (yellow in original).

FIG. 5 is an exemplary clinical decision tree of ovarian cancer, indicating key sites for utility of tumor reactive IgG as biomarkers.

FIG. 6 is an image of 2-DIGE image of tumor-derived antigens recognized by patients with ovarian cancer. Spots 1, 17-21, 28-32, and A-C (green in original) are antigenic proteins recognized by IgG from ovarian cancer patients with Stage I disease. Spots 2-16 and 22-27 (red in original) are antigenic proteins recognized by IgG from ovarian cancer patients with Stage III disease. The brightest spots (at top of image, just left of center; yellow in original) are antigens recognized by both.

FIG. 7 shows a representative comparison of immunoreactive spots using sera from ovarian cancer patients with Stage I disease or Stage III disease. Upper panels present the 2-D gels results with the specific spots indicated. The lower panel presents the quantifications of those spots.

FIGS. 8A-B are each six box-whisker plots showing the results of quantitation of dot immunoblots presenting the immunoreactivity of sera from normal controls (n=40), women with benign disease (n=40) and patients with various stages of ovarian cancer (Stage I, n=35; Stage II, n=25; Stage III, n=40; Stage 1V, n=25) patients against nucleophosmin, cathepsin D, glucose regulated protein 78 (GRP78), homeobox A7 (HoxA7), heat shock protein 90 kDa (HSP90), mucin 16, cell surface associated (Muc16), NY-ESO-1 (also known as cancer/testis antigen 1B), p53, alkaline phosphatase, placentall (PLAP), SSX common antigen, survivin, and Tumor-Associated Glycoprotein 72 (TAG-72). * denotes significantly different (p<0.05) than either control or benign.

FIG. 9 is a set of six box-whisker plots showing the results of quantitation of dot immunoblots presenting the immunoreactivity of sera from normal controls and women with pancreatic (n=15), lung (n=15), breast (n=15), colon (n=15), and ovarian cancers (n=40). * denotes significantly different (p<0.05) than control.

### DETAILED DESCRIPTION

As described herein, sera from ovarian cancer patients exhibited significantly greater immunoreactivities than either controls or women with benign disease. While late stage patients recognized more proteins at greater intensity, stage-specific differential recognition patterns were observed in the IgG subclasses, with the greatest recognition appearing in IgG2 subclasses. Immunoreactivity in IgG2 and IgG3 from stage I and II patients appears to be most intense with nuclear antigens >40 kD, while, in stage III patients, additional immunoreactivity was present in the <40 kD components. Stage III patients also exhibited similar reaction with membrane antigens <40 kD. 2D-electrophoresis revealed 32 stage-linked antigenic differences with 11 in early stage and 21 in late stage ovarian cancer. Thus, due to the timing and stability of humoral responses, quantitation of IgG subclasses recognizing specific tumor antigens provides superior biomarkers for early cancer identification and allows for differentiation of benign versus malignant ovarian masses and early and late stage cancers.

The concept of immunosurveillance suggests that immune cells scan host cells for mutations, which when detected, the mutated cells are eradicated. While significant literature alludes to cancer being associated with an immunosuppressed state, it may be more accurate that there is a Th1 to Th2 shift associated with the development of cancer. Activation of Th2 cells is an antigen-specific mechanism of immunodepression, since Th2 cells have extreme sensitivity to the antigen by means of the T-cell receptor. In general, Th2-driven antibody responses recognizing tumor components are non-protective and may contribute to tumor progression by inhibiting the Th1 cell-mediated immune response (Clerici M, Clerici E, Shearer G M, J Natl Cancer Inst 1996, 88(7):461-2). In contrast to the “strong” Th1 immune response generated by transplantable tumors, spontaneous tumors elicit a quantitatively mild Th2 immune response. Thus, spontaneous tumors may not stimulate an appropriate immune response but rather elicit non-protective humoral immune responses that are not adequate for tumor eradication. Specifically, a “weak” immune response is a state in which immunological recognition of the tumor occurs but eradication is not achieved.

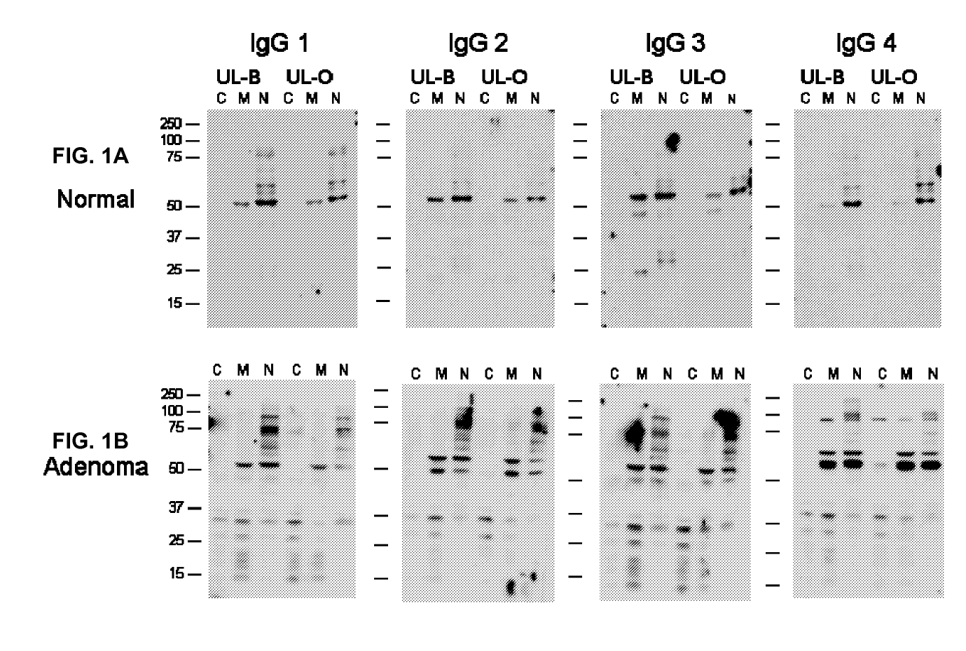

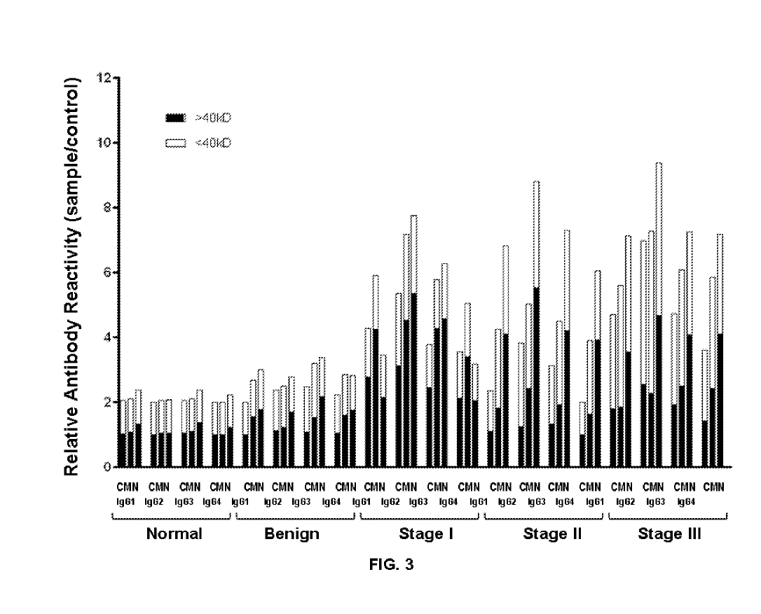

While the induction of the non-protective humoral response in ovarian cancer fails to eradicate the tumor, the appearance of these tumor-reactive immunoglobulins, linked with this Th2 shift, appears to be characteristic of cancer and thus, may have utility as biomarkers of events associated with ovarian cancer development. Although the appearance of specific proteins in the circulation can be indicative of cancer, their additional association with non-cancerous pathologies diminishes their specificity and prevents their use in cancer screening. Using a broad array of tumor-derived antigens, specific immunoreactivity has been detected in all cancer patients evaluated (Taylor D D, Homesley H D, Doellgast G J., Am J Reprod Immunol 1984, 6:179-184; Marx D, Frey M, Zentgraf H, Adelssen G, Schauer A, Kuhn W, Meden H., Cancer Detect & Prevent 2001, 25:117-22); however, recognition of these proteins by antibodies from non-cancer-bearing volunteers is a rare (<1%) event (Pfreundschuh M., Cancer Chemother Pharmacol 2000, 46 (suppl):S3-S7). Thus, the induction of humoral responses against tumor-derived proteins appears to enhance cancer specificity. In our assay system, only minor levels of tumor-reactive antibodies were detected in non-cancer bearing controls or women with benign ovarian disease (FIG. 1). In our previous publications, we addressed the reactivity of patient-derived immunoglobulins with antigens isolated from normal ovary (Gercel-Taylor C, Bazzett L B, Taylor D D., Gynecol Oncol 2001, 81:71-76; Taylor D D, Gercel-Taylor C., Oncol Rep 1998 November-December, 5(6):1519-24; Taylor D D, Homesley H D, Doellgast G J., Am J Reprod Immunol 1984, 6:179-184; Taylor D D, Gercel-Taylor C, Parker L P., Gynecol Oncol 2009, 115:112-120). These studies demonstrated the recognition of one to two bands within the normal ovarian antigen population. Due to the design of the analyses, it is not possible to define whether the background reactivity observed in normal controls represents binding via the Fc or Fab portion of the antibody. Regardless, only a few (2-4) components are weakly recognized in the membrane and nuclear antigens and no significant differences were observed among IgG subclasses (FIGS. 1A and 3). Additional membrane and nuclear antigens were weakly recognized by IgGs from patients with benign ovarian disease (FIGS. 1B and 3), with the reactivity significantly greater within the IgG3 subclass.

In our original report, demonstrating the presence of tumor-reactive immunoglobulins in ovarian cancer patients and establishing the foundation of autologous serological typing of cancer antigens (Taylor D D, Doellgast G J., Analytical Biochem 1979, 98:53-59), the predominant antibody was shown to be of the IgG class. IgG is the major effector molecule of the humoral immune response, accounting for approximately 75% of the total immunoglobulins in the circulation, expressing their activity during a secondary antibody response. The human IgG compartment consists of four distinct subclasses, designated IgG1, IgG2, IgG3 and IgG4 and their mean serum concentrations are 6.98 mg/ml for IgG1, 3.80 mg/ml for IgG2, 0.51 mg/ml for IgG3, and 0.56 mg/ml for IgG4. The principal biological activities of IgGs are related to their effector functions, including activation of complement and binding Fc receptors to mediate antibody-dependent cellular cytotoxicity. Although their heavy chains exhibit >95% sequence homology, IgG subclasses express unique profiles of effector activities (Ravetch T V, Bolland S. IgG Fc receptors. Annual Rev Immunol 2001; 19:275-290). In general, protein antigens characteristically elicit IgG1 and IgG3 responses and these isotypes are capable of activating all Fc receptors and the Cl component of complement. Induction of IgG4 subclass appears to be characteristic of chronic antigen stimulation, such as observed in autoimmune disease; IgG4 exhibits restricted Fc receptor activation and does not activate Clq. The IgG2 subclass often predominates in responses to carbohydrate antigens and also exhibits restricted Fc receptor and Clq activation (Ravetch J V, Bolland S. IgG Fc receptors. Annual Rev Immunol 2001; 19:275-290). For example, in acute lymphocytic leukemia (ALL), elevated levels of 9-O-acetylated sialic acid (AcSA)-specific IgG2 were induced, which were unable to trigger activation of Fc receptors, the complement cascade and cell-mediated cytotoxicity, although its glycotope-binding ability was unaffected (Bandyopadhyay S, Bhattacharyya A, Mallick A, Sen A K, Tripathi G, Das T, Sa G, Bhattacharya D K, Mandal C., Intl Immunol 2005, 17:177-191). The subclass switching of anti-9-OAcSA to IgG2, in ALL, was linked with alterations in the tumor cell's glycosylation profile. In patients with non-hematologic tumors responding to daily low-dose IL-2, a significant decrease in mean IgG2 levels were observed; however, no significant changes were seen in IgG1, IgG3, or IgG4 levels (Soiffer R J, Murray C, Ritz J, Phillips N, Jacobsohn D, Chartier S, Ambrosino D M., Blood 1995, 85:925-928).

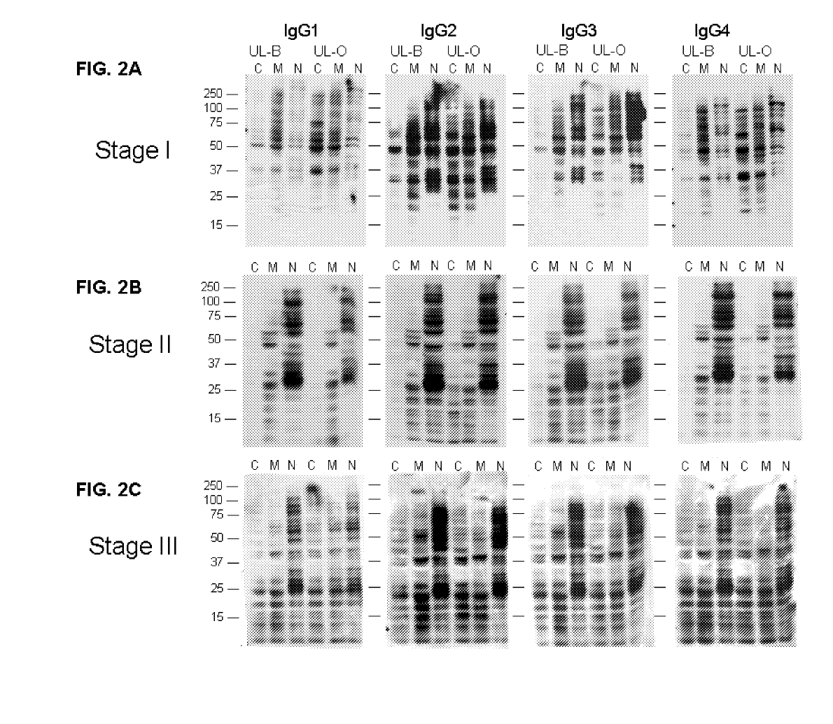

In our study, all of the ovarian cancer patients evaluated exhibited the greatest reactivity with tumor-derived antigens in the IgG2 subclass (FIGS. 2 and 3). In patients with Stage I and II disease, this IgG2 response was directed primarily at nuclear antigens greater than 40 kD. In Stage I disease, these nuclear antigens were only weakly recognized by IgG1 and IgG4. Our previous work, as well as that of others, have indicated that the “immunodepressed” state of ovarian cancer patients is biphasic; in early stage disease, the suppression appears to be antigen-specific, while in late stage disease, it is non-specific (Taylor D D, Homesley H D, Doellgast G J., Cancer Res 1980; 40: 4064-4069). Our current results support this observation, as patients with Stage III disease exhibit broad, similar recognition patterns in all IgG subclasses, even though IgG2 still exhibits a significantly greater level of the tumor reactivity.

The analyses of serologically defined biomarkers have generally focused on patient extremes; advanced stage cancer versus completely normal volunteers. Based on these extremes, many biomarkers exhibit the specificity and sensitivity necessary for utility in screening and diagnosis. However, when benign disease, pre-malignant disease, early stage cancer and inflammatory pathologies are included, most biomarkers fail to reach adequate sensitivity and specificity for clinical utility. Jacobs and Menon calculated that to be an effective screening test, an assay needs to achieve a minimum of 99.6% specificity (Jacobs I J, Menon U., Mol Cell Proteomics, 3:355-366, 2004). Recently, global epitope/antigen profiling using serum antibodies as analytes has been extensively investigated as diagnostic markers, particularly in high risk populations (Chatterjee M, Wojciechowski J, Tainsky M A., Mol Biol 2009, 520:21-38; Chatterjee M, Mohapatra S, Ionan A, Bawa G, Ali-Fehmi R, Wang X, Nowak J, Ye B, Nahhas F A, Lu K, Witkin S S, Fishman D, Munkarah A, Morris R, Levin N K, Shirley N N, Tromp G, Abrams J, Draghici S, Tainsky M A., Cancer Res 2006, 66:1181-1190). This combination of high-throughput selection and array-based serologic detection of recombinant antigens using a phage display system, defines large panels of epitopes or tumor antigens in an unbiased fashion without regard to function (Chatterjee M, Wojciechowski J, Tainsky M A., Mol Biol 2009, 520:21-38). Chatterjee et al (Chatterjee M, Mohapatra S, Ionan A, Bawa G, Ali-Fehmi R, Wang X, Nowak J, Ye B, Nahhas F A, Lu K, Witkin S S, Fishman D, Munkarah A, Morris R, Levin N K, Shirley N N, Tromp G, Abrams J, Draghici S, Tainsky M A., Cancer Res 2006, 66:1181-1190) identified 65 different antigens and demonstrated reactivity in sera from 32 ovarian cancer patients and no reactivity in sera from healthy female controls and 14 patients with either benign disease or other malignant gynecologic diseases. Despite the high-throughput capacity of this technique, it possesses several limitations, including high cross-reactivity with bacterial or phage components, co-expression of cDNA derived from normal tissue (including lymphoid cells) present within the original tumor and an absence of cancer-linked post-translational modifications and processing, which can result in loss of immunoreactivity of these “engineered” protein targets (Nishikawa H, Tanida K, Ikeda H, Sakakura M, Miyahara Y, Aota T, Mukai K, Watanabe M, Kuribayashi K, Old U, Shiku H., Proc Natl Acad Sci USA 2001, 98:14571-14576). Using their 65 antigen array, Chatterjee et al (Chatterjee M, Mohapatra S, Ionan A, Bawa G, Ali-Fehmi R, Wang X, Nowak J, Ye B, Nahhas F A, Lu K, Witkin S S, Fishman D, Munkarah A, Morris R, Levin N K, Shirley N N, Tromp G, Abrams J, Draghici S, Tainsky M A., Cancer Res 2006, 66:1181-1190) found only a sensitivity and specificity of 55% and 98%, respectively.

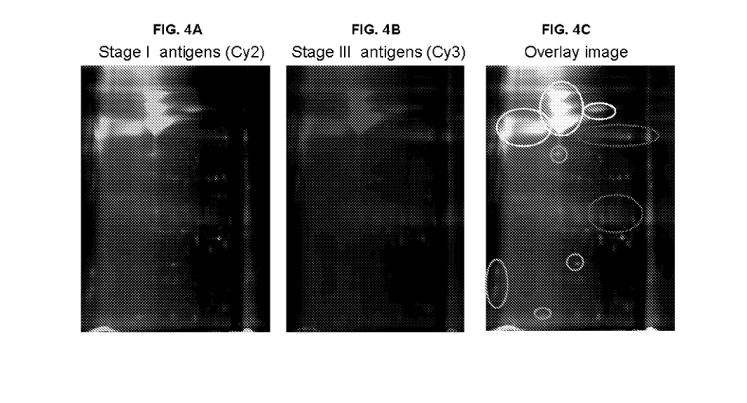

The stated goal of many recent biomarker studies has been the early detection of cancer, irrespective of stage (Singh A P, Senapati S, Ponnusamy M P, Jain M, Lele S M, Davis J S, Remmenga S, Batra S K., Lancet Oncol 2008, 9: 1076-1085). Many of the “new” biomarkers for ovarian cancer either lack cancer specificity or are linked with advanced stage disease (Kim K, Visintin I, Alvero A B, Mor G., Clin Lab Med 2009, 29:47-55). The key to improving cancer survival is the detection of early stage cancers, since the five-year survival rate is significantly improved to 93 percent if the cancer is diagnosed at Stage I. Unfortunately, only 19 percent of ovarian cancer cases are diagnosed at this stage. Despite advances in surgery and chemotherapy, the majority of patients with advanced ovarian cancer will recur within a median of 12-18 months after completing first-line therapy. The risk of recurrence varies based on several factors, including the stage at diagnosis, with approximately 90-95% of stage 1V patients recurring. Further, 80-85% of stage III patients who are suboptimally debulked will recur, as will 70-80% of stage III patients optimally debulked and 30% of stage II patients. In contrast, less than 10% of stage I patients will recur. Recurrent ovarian cancer is invariably fatal and treatment of recurrent disease is palliative and is generally initiated with the goals of controlling disease-related symptoms, limiting treatment-related toxicity, maintaining quality of life, and prolonging survival (Herzog T J., Clin Cancer Res 2004, 10:7439-7449). Thus, to have clinical utility, it is critical that a biomarker is capable of identifying early stage disease with specificity greater than 99.6%. The immunoreactivity of IgG2 and IgG3 with nuclear and membrane antigens with all stages of ovarian cancer were greater than that observed for controls or patients with benign disease (FIGS. 2 and 3). In addition to the greater reactivity seen in advanced cancer patients, assessment of the cellular antigens recognized identified both shared and stage specific antigens (FIG. 4 and Table 1). Thus, recognition by patient-derived IgG2 of stage-specific proteins can define both the presence and stage of ovarian cancer. The lack of effector functions within the IgG2 subclass may be an important contributor to the immunosuppressive environment associated with progressive cancer. The IgG2 can block cellular recognition of specific antigens, as well as remove targetable antigens from the cell surface. Another key issue for Gynecologic Oncologists is differentiating ultrasound-identified benign versus malignant ovarian masses. As shown in FIGS. 1 and 2, the presence of IgG2 reactive with tumor derived nuclear antigens can distinguish benign adenoma and Stage I ovarian cancer.

These findings highlight one embodiment of the diagnostic methods described herein, which can consist of a two-tiered assay. As previously demonstrated, many of the antigenic proteins defined by patient humoral responses appear to define the presence of cancer and can differentiate these from benign ovarian disease versus normal (Taylor D D, Gercel-Taylor C, Parker L P., Gynecol Oncol 2009, 115:112-120), which would constitute the first-tier of a screening assay or an assay to differentiate the nature of ovarian masses. As a second tier, specific antigenic proteins derived from the cytosol, nuclear and membrane compartments exhibit stage specificity (See FIG. 4 and Table 1). Among the initial 12 antigens whose recognition was compared, only PLAP exhibited ovarian cancer specificity; however, our analyses indicated the presence of other ovarian specific antigens and these are currently being identified (Taylor D D, Gercel-Taylor C, Parker L P., Gynecol Oncol 2009, 115:112-120). The recognition patterns of these proteins by patient-derived IgG2 and IgG3 can define the stage of ovarian cancer.

In addition, due to their early appearance during neoplastic development and stability of IgG in the peripheral circulation, quantitation of IgG subclasses recognizing specific tumor antigens provides superior biomarkers for identification of early cancers and allows for differentiation of benign versus malignant ovarian masses.

Methods of Use

Included herein are methods for diagnosing and staging cancer, e.g., pancreatic, lung, breast, colon, or ovarian cancer, based on detecting the presence of autoantibodies specific for tumor-associated antigens as described herein. The presence and/or level of a protein can be evaluated using methods known in the art, e.g., using quantitative immunoassay methods. In some embodiments, high throughput methods, e.g., protein or gene chips as are known in the art (see, e.g., Ch. 12, Genomics, in Griffiths et al., Eds. Modern genetic Analysis, 1999; W. H. Freeman and Company, Ekins and Chu, Trends in Biotechnology, 1999, 17:217-218; MacBeath and Schreiber, Science 2000, 289(5485):1760-1763; Simpson, Proteins and Proteomics: A Laboratory Manual, Cold Spring Harbor Laboratory Press, 2002; Hardiman, Microarrays Methods and Applications: Nuts & Bolts, DNA Press, 2003), can be used to detect the presence and/or level of proteins.

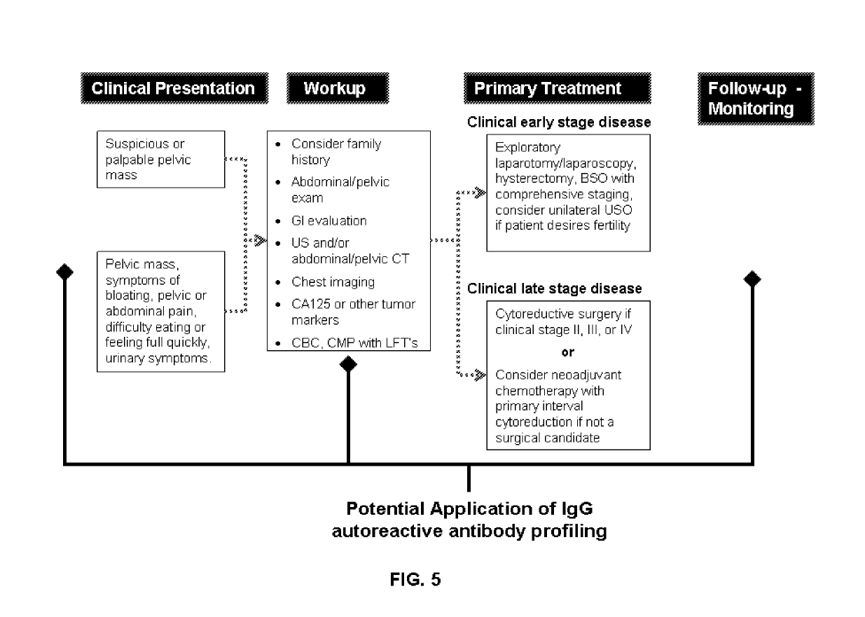

In the exemplary clinical decision tree of ovarian cancer (FIG. 5), there are three potential points for applying biomarkers, such as tumor-reactive antibodies. First, potential biomarkers can be used for screening by applying them to the detection of cancer in asymptomatic individuals in high risk populations or in the general population. Second, potential biomarkers can be used for definitive diagnosis of individuals with suspicious or palpable masses, ultrasound-identified masses or symptoms of pelvic or abdominal pain. Third, potential biomarkers can be used for disease monitoring or follow-up in individuals treated for ovarian cancer (by surgery and first-line chemotherapy) to assess the therapeutic responses of residual and metastatic disease and for early identification of recurrence. The biomarkers described herein can be use at one or more, or all three, of the above, or at other time points, e.g., as determined by a health care provider or insurance provider.

Although ovarian cancer is used as an example herein, the methods described herein can also be used for other cancers, e.g., other cancers of epithelial origin, e.g., carcinomas, e.g., pancreatic, lung, breast, or colon cancer.

### EXAMPLES

The invention is further described in the following examples, which do not limit the scope of the invention described in the claims.

### Example 1

### Recognition of Specific Antigenic Targets by Tumor Reactive IgG

Using target proteins derived from specific compartments of ovarian tumor cells, the presence of humoral responses was assessed in normal female controls and women with benign ovarian disease.

The experiments were performed on banked sera obtained from the Gynecologic Oncology Group Serum Bank (Columbus, Ohio). Sera included specimens from patients with serous papillary adenocarcinoma diagnosed at Stage I (n=15), stage II (n=15), and stage III (n=20) or benign ovarian disease (serous adenoma, n=15) and from age-matched female volunteers (n=20). Sera from ovarian patients and patients with benign ovarian disease were obtained pre-surgery. The control group consisted of age-matched healthy females (no diagnosis of any cancer, not genetically predisposed for ovarian or breast cancer, and disease-free at least 6 months after sample collection), undergoing routine gynecologic examinations. All sera are stored at −70° C. Age, pathologic diagnosis, and histological analyses at the time of sample acquisition were obtained for all groups. The age differences were not significant, with the mean age of the non-tumor-bearing controls being 57.0±4.1 years, compared to 58.1±5.2 for patients with ovarian cancer and 56.9±5.3 years for patients with benign disease. Although CA125 has been reported to perform poorly as a diagnostic marker, pre-surgery CA125 levels were obtained from patient records. Of the 20 control subjects with no evidence of cancer, 5 (25%) exhibited CA125 levels >35 units/ml, while 7 of the 15 women (47%) with benign ovarian masses exhibited elevated CA125 (>35 units/ml). Of the 15 patients with Stage I disease, 9 exhibited CA125 levels <35 units/ml with a mean of 26.74±16.22. Of the 20 patients with Stage III ovarian cancer, 18 women expressed elevated levels of CA125 with a mean level of 142.12±82.33 units/mL (ranging from 42-937).

Humoral responses were assessed by western immunoblot. Tumor derived cellular proteins were prepared for western blot analysis as follows. Total cellular proteins, including those from cellular compartments, were isolated from human ovarian tumor cell lines established in our laboratory from women with Stage Mc cyst adenocarcinoma of the ovary (designated UL-B and UL-O). UL-O cells were derived from a 48-year old Caucasian woman with a family history of breast/ovarian cancers (medical records indicated that the patient was BRCA1+), while UL-B was derived from 72 year old Caucasian woman, with no family history of cancer (Taylor D D, Gercel-Taylor C., Gynecol Oncol 2008, 110:13-21). These ovarian tumor cells are grown in RPMI 1640 medium supplemented with 10% fetal bovine serum, 0.1 mM nonessential amino acids, 1 mM sodium pyruvate, 200 mM L-glutamine, 100 mg/ml streptomycin and 100 IU/ml penicillin in a humidified 5% CO2 atmosphere. Cell viability was evaluated by trypan blue exclusion and all cultures utilized for this study were >95% viable.

Subcellular fractionations of proteins were performed according to the manufacturer's instructions (BioVision, Mountain View, Calif.). Tumor cell monolayers were extensive washed with 20 mM sodium phosphate buffered saline (PBS), then removed from the monolayer by scraping and separated into fractions derived from the cytosol, membrane, and nucleus. The protein concentrations of each cellular fraction were determined using the Bradford microassay (Bio-Rad Laboratories, Hercules, Calif.). To assess appropriate fractionation, proteins markers of the membrane (placental type alkaline phosphatase and EpCAM), nuclear (histone H3) and cytoplasmic (GAPDH) fractions are evaluated by western immunoblotting. Antibodies were obtained from Santa Cruz Biotechnology and were anti-PLAP (sc-47691), anti-EpCAM (sc-73491), anti-histone H3 (sc-10809) and anti-GAPDH (sc-47724). Each subcellular fraction exhibited detectable bands on immunoblots only for their specific marker.

To visualize patient autoantibody reactivity patterns, solubilized proteins (40 μg/lane) were applied to a 10% SDS-PAGE gel, electrophoretically separated (Laemmli UK., Nature 1970, 227, 680-685) and analyzed by western immunoblotting (Brown R, Clugston C, Burns P, Edlin A, Vasey P, Vojtesek B, Kaye S B., Int J Cancer 1993, 55, 678-684). Nitrocellulose membranes were blocked using SuperBlock (Pierce Chemical) for 3 hours and probed overnight at 4° C. with patient sera, diluted 1:100 in TBS containing 5% non-fat dried milk with Tween 20, followed by peroxidase-conjugated anti-human IgG1, IgG2, IgG3, or IgG4 (AbD Serotec, Raleigh, N.C.). We previously determined the 1:100 dilutions for this test, using serial dilutions of patient and control sera to identify the optimal dilution to distinguish seropositive patients and seronegative controls (Taylor D D, Homesley H D, Doellgast G J., Am J Reprod Immunol 1984, 6:179-184). Bound immune complexes were visualized by enhanced chemiluminescence (ECL, Amersham Life Sciences, Arlington Heights, Ill.). The resulting x-ray film was scanned, digitized and converted into pixel density using Un-scan-it software (Silk Scientific Corp., Orem, Utah) Immunoreactivities for antigens, either greater or less than 40 kD, from each cellular compartment were standardized using the pixel values of a control standard (HRP-anti-mouse Ig) included on each gel. The standardized pixel values were divided by the negative control lane on each gel, such that a lane with no immunoreactivity exhibits a value of 1. Duplicate gels were run for each patient and the resulting ratios from these gels were averaged. The mean values and standard deviations were calculated from the averages of all patients within each stage.

2D DIGE (2-dimensional difference in gel electrophoresis) protein expression profiling was performed as follows. The immunoaffinity-isolated cellular proteins (300 μg) eluted from Stage I patients are labeled with Cy2, while proteins (300 μg) eluted from columns with Stage III patient-derived IgG were labeled with Cy3. The two samples were simultaneously separated on a single 2D gel, using isoelectric focusing (IEF) in the first dimension and SDS polyacrylamide gel electrophoresis (SDS-PAGE) in the second dimension. After electrophoresis, the gel was scanned using a Typhoon image scanner. Each scan revealed one of the CyDye signals (Cy2 and Cy3). ImageQuant software was used to generate an image presentation data including the single and overlay images. The images were then subjected to DeCyder software analysis, which automatically located and analyzed multiplexed samples. The 2D gel spots were removed, washed to remove staining dye and inhibitory chemicals and dried to absorb maximum volume of digestion buffer. The dried 2D gel spots were rehydrated in digestion buffer containing sequencing grade modified trypsin (1:30 by mass) and proteins were digested in-gel at 37° C. Digested peptides were extracted from gel with trifluoroacetic acid extraction buffer and the digested tryptic peptides were desalted using C-18 Zip-tips (Millipore). The desalted peptides were mixed with CHCA matrix (α-cyano-4-hydroxycinnamic acid) and spotted into wells of a MALDI plate. Mass spectra (MS) of the peptides in each sample were obtained using an Applied Biosystems 4700 Proteomics Analyzer. A minimum of 10 of the most abundant peptides for each sample were further subjected to fragmentation and tandem mass spectrometry (MS/MS) analysis. Protein identification was based on peptide fingerprint mass mapping and peptide fragmentation mapping (using MS/MS spectra). Combined MS and MS/MS spectra were submitted for database search using GPS Explorer software equipped with the MASCOT search engine to identify proteins from primary sequence databases.

The recognition of specific protein spots by each patient was stratified by the presence or absence of antibodies reactive with each specific antigen. Comparisons between the presence of specific antigens from non-cancer bearing controls and patients at each stage of ovarian cancer were performed by the Kruskal-Wallis test. Tests with p<0.05 are considered statistically significant.

The results are shown in FIGS. 1A-1B. The control samples failed to exhibit any reactivity with antigens derived from the cytosol, regardless of IgG subclass. The controls recognized a single band at 50 kD in antigens derived from the membrane compartment in all IgG subclasses. These controls also recognized the 50 kD antigen in the nuclear fraction, as well as two bands at 52 and 60 kD in all IgG subclasses. The IgG3 subclasses also exhibit additional weak reactive bands. In patients with benign disease, the 50 kD band is recognized in the membrane and nuclear fractions with all IgG subclasses. An additional 34 kD band is recognized in all fractions with all IgG subclasses. A group of bands between 15-30 kD was recognized in the membrane fraction by IgG1, IgG3 and IgG4, with an additional group between 55-100 kD being recognized in the nuclear fraction by all IgG subclasses.

The presence of humoral immune responses was then analyzed using antibodies derived from women with Stage I (FIG. 2A), Stage II (FIG. 2B) and Stage III (FIG. 2C) ovarian cancer using protein isolated from tumor cells lines. Previous studies demonstrated that cancer patient sera exhibit reactivity with only a few minor bands in normal ovarian epithelium (Taylor D D, Gercel-Taylor C, Parker L P., Gynecol Oncol 2009, 115:112-120). Differences in the antigen recognition were observed for ovarian tumor derived antigens. The 50 kD band recognition appears to be shared by all patients.

For Stage I patients (FIG. 2A), western immunoblots identified multiple bands ranging in molecular weight from 20 to 140 kD. This immunoreactivity was IgG2>IgG3>IgG1=IgG4 (FIG. 3). For IgG2 and IgG3, reactivity was greater for nuclear antigens, followed by membrane antigens and minor recognition of cytosol antigens. In contrast, for IgG1 and IgG4, membrane antigens exhibited the greatest reactivity followed by cytosol antigens, with the least reactivity with nuclear antigens. The primary immunoreactivity was observed in antigens exhibiting molecular weights greater than 40 kD.

For patients with Stage II ovarian cancer, western immunoblots identified multiple bands ranging in molecular weight from 10 to 140 kD (FIG. 2B). This immunoreactivity was IgG2>IgG3>IgG1>IgG4 (FIG. 3). For all IgG subclasses, reactivity was greater for nuclear antigens, followed by membrane antigens and minor recognition of cytosol antigens. For IgG1 and IgG4, no significant reactivity was observed by antigens derived from the cytosol, while for IgG2 and IgG3, lower reactivity (2.68±1.25 and 1.85±0.81, respectively) was observed primarily in the less than 40 kD antigens. Patient-derived IgG primarily recognized <40 kD antigens from the membrane compartment with similar reactivity in all subclasses (for IgG1=2.59±0.69, IgG2=2.79±0.63, IgG3=2.61±0.71, and IgG4=2.38±0.56).

For Stage III patients, western immunoblots identified multiple bands ranging in molecular weight from 10 to 140 kD (FIG. 2C). This immunoreactivity was IgG2≧IgG3=IgG4>IgG1 (FIG. 3). Except for intensity, the recognition patterns appeared to be identical for all IgG subclasses. For IgG2 and IgG3, reactivity was slightly greater for nuclear antigens, followed by membrane antigens and less recognition of cytosol antigens. For nuclear antigens, the level of reactivity was similar for the <40 kD and >40 kD antigens for IgG2 (4.68±0.63 versus 4.71±0.72, respectively) and IgG3 (3.42±0.58 versus 4.08±0.67, respectively). For membrane antigens, the primary reactivity was observed with antigens less than 40 kD: IgG1 exhibited a 2.08-fold greater reactivity with <40 kD antigens (versus >40 kD antigen), IgG2 exhibited a 2.27-fold greater reactivity, IgG3 exhibited a 1.64-fold greater reactivity, and IgG4 exhibited a 1.51-fold greater reactivity. Antigens derived from the cytosol compartment exhibited a similar greater reactivity with the <40 kD antigens.

### Example 2

### Identification of Immunoreactive Components

The identity of antigens mediating the patient IgG recognition was examined using 2-DIGE (FIGS. 4A, B, C). Antigens recognized by patients with benign ovarian disease were removed by immunoabsorption using affinity columns constructed with benign patient-derived IgG prior to analyzing cancer patient reactivity.

To prepare immunoaffinity columns from patients' sera to isolate immunoreactive proteins, patients' sera (0.5 ml) were diluted with 0.5 ml of binding buffer (20 mM sodium phosphate, pH7.0) and applied to 1 ml HiTrap Protein G columns (GE Healthcare BioSciences, Piscataway, N.J.). The sample was recirculated on the column for 1 hour at room temperature and then washed with 10 ml of binding buffer or until no material absorbing at 280 nm appeared in the effluent. The patient-derived IgG bound to the HiTrap column was then cross-linked to the Protein G. To crosslink, the column was equilibrated with 0.2M triethanolamine, pH8.0 and 1 ml cross-linking buffer containing 25 mM DMP (dimethyl pimelidate dihydrochloride) was added and incubated at room temperature for 45 minutes. Then, 1 ml blocking buffer (0.1M ethanolamine, pH 8.2) was added to the column and incubated for 1 hour at room temperature. The immunoaffinity column was washed twice with binding buffer, followed by addition of 1 ml 0.1M glycine-HC1 (pH 2.5) to elute antibody not cross-linked with DMP.

To separate stage-associated immunoreactive proteins, monolayers of UL-B cells were washed extensive with PBS and then removed by scraping. The cells were resuspended in ice-cold PBS and centrifuged at 400×g for 10 minutes. The cell pellet was lysed in 1% NP-40, 500 mM NaC1, 50 mM Tris (pH7.5), 1 mM DTT, and cocktails of protease and phosphatase inhibitors (Sigma-Aldrich, St. Louis, Mo.) and this suspension centrifuged at 10,000×g for 15 minutes. Supernatants were clarified by incubation with anti-human IgG,A,M-agarose for 1 hour. After centrifugation at 3000 rpm, clarified cell lysates were use to identify specific immunoreactivity. These solubilized cellular proteins were applied to immunoaffinity columns constructed of pooled patient-derived IgG. Initially, the cellular proteins were pre-absorbed using an affinity column constructed on IgG from patients with benign disease. The non-binding proteins were either applied to affinity columns constructed of IgG from Stage I patients or from Stage III patients. These proteins were recirculated on the columns for 1 hour at room temperature and then washed with 10 ml of binding buffer (20 mM sodium phosphate, pH7.0) or until no material, absorbing at 280 nm, appeared in the effluent. Then bound material was eluted from the column by addition of 1 ml 0.1M glycine-HC1 (pH2.5), monitoring the absorbance at 280 nm. The peak of eluted protein from each immunoaffinity column was collected and the pH neutralized by addition of Tris base. Protein concentrations for the eluted proteins were determined by the Bradford microassay.

The proteins, eluted from IgG of Stage I patients, were labeled with Cy2 and those from stage III were labeled with Cy3. After 2-D separation, analyses of the multiplexed samples were performed to define shared and unique spots (FIG. 4C). This analysis revealed 32 stage-linked antigenic differences. Of these unique patterns, 11 proteins exhibited increased recognition by Stage I patients (greater than 4-fold), while 21 proteins exhibited increased recognition by Stage III patients. Of these, using peptide fingerprint mass mapping and peptide fragmentation mapping (using MS/MS spectra), the identities of six shared and 5 stage-specific proteins for each stage I and III were determined (Table 1).

TABLE 1
Mass spectrometric identification of spots from
the 2-DIGE of tumor-derived antigens recognized
by Stage I patients, Stage III patients or both.

| | |
|---|---|
| Nuclear | Cytoplasmic |

Shared recognition

| | |
|---|---|
| Nucleophosmin (B23) | Glucose regulated protein |
| SSX-2 | (GRP) 78 (in ER) |
| SSX-4 | proCathepsin D (in lysosomes) |
| HoxAV | |

Early recognition (Stage I)

| | |
|---|---|
| HNRNP A2/B1 | Pyridoxal kinase |
| NME1/NME2 | Galectin-1 |
| | HSP90 |

Late recognition (Stage III)

| | |
|---|---|
| Zinc finger DHHC-type | Peroxiredoxin |
| containing 7, isoform 2 | |
| Survivin | |
| p53 | |
| p73 | |

As shown in Table 1, within antigens preferentially recognized by Stage I patients, heterogeneous nuclear ribonucleoprotein (HNRNP A2/B1) and non-metastatic cells 1/non-metastatic cells 2 (NME1/NME2) are localized to the nucleus, while pyridoxal kinase, galectin-1 and heat shock protein 90 are generally expressed in the cytosol. Within antigens preferentially recognized by Stage III patients, zinc finger DHHC-type containing 7 isoform 2, survivin, p53 and p73 are localized to the nucleus and peroxiredoxin is present in the cytosol. With proteins exhibiting recognition by both groups of patients, nucleophosmin (B23), synovial sarcoma X breakpoint proteins (SSX2, SSX4), and HoxA7 represent proteins generally localized to the nucleus, while glucose regulated protein 78 is localized to the endoplasmic reticulum and proCathepsin D is present in the lysosome.

### Example 3

### Differential Recognition of Tumor-Cell Derived Antigens

To define the differential recognition of tumor cell derived antigens, solubilized cellular antigens from UL-6 were isolated based on their specific recognition by IgG from patients with early or late stage ovarian cancer. IgG from these patients was isolated on a protein G-Sepharose column and crosslinked using DMP. In a similar fashion, IgG from women with benign ovarian disease was also isolated and coupled to Protein G. Initially, the solubilized proteins were applied to the immunoaffinity column derived from benign disease. This removed those proteins reacting with IgG from these patients. The non-binding proteins were then applied to either the immunoaffinity columns prepared with early or late stage derived IgG. The columns were extensively washed and then eluted. The eluted proteins were compared by 2-DIGE. The reactive spots isolated from early stage patients was compared with late stage patients by overlaying the digitized images. The reactive spots were then quantified and spots expressing a 4-fold increase/decrease were defined by MS sequencing.

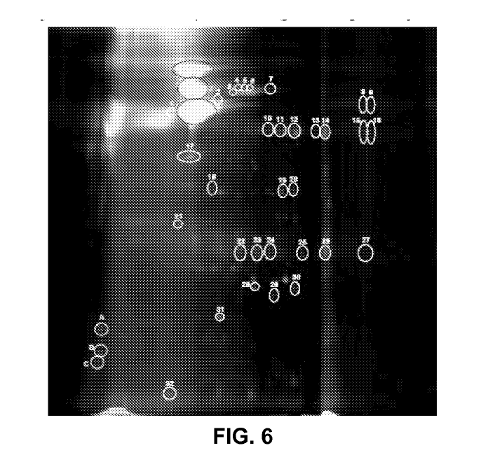

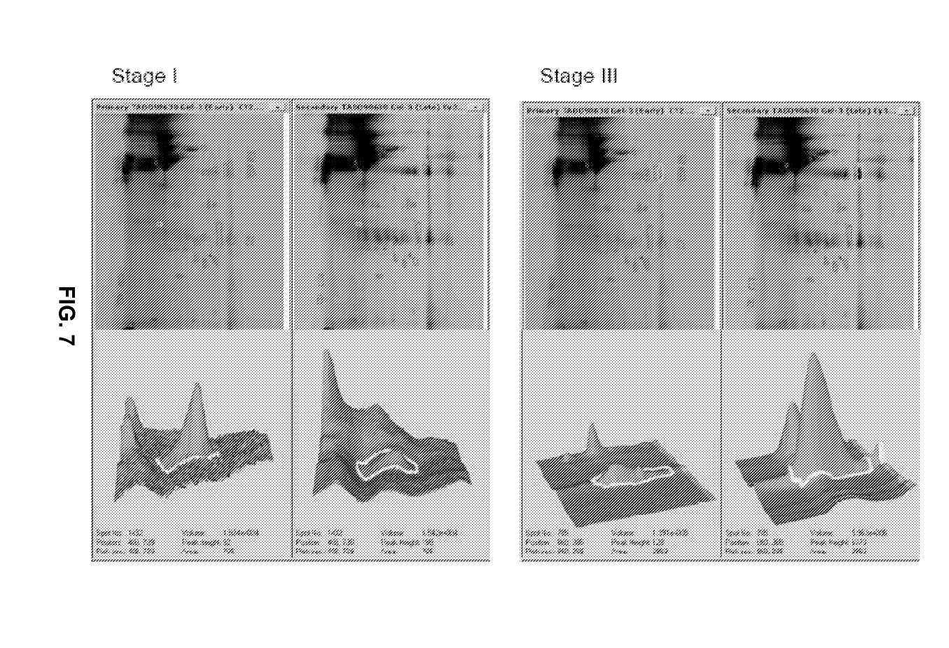

The results are shown in FIGS. 6 and 7. A total of 32 reactive spots were identified. Reactive spots include nucleophosmin, nucleoside diphosphate kinase, NME1-NME2, nuclear riboprotein A2/B1, Zn-DHHC-containing 7, and aldose reductase isoforms.

### Example 4

### Reactivity in Non-Ovarian Cancers

Specific immunoreactive proteins were isolated from cultured cells by immunosorbent chromatography. Commercial antibodies for each protein were obtained: anti-proCathepsin D (rabbit polyclonal, Calbiochem), ant-GRP78 (goat polyclonal, Santa Cruz Biotechnology [SCBT]), ant-p53 (mouse monoclonal, Abcam), anti-nucleophosmin (mouse monoclonal, Abcam), anti-placental alkaline phosphatase (mouse monoclonal, Abeam), anti-SSX common epitope (rabbit polyclonal, SCBT), anti-survivin (rabbit polyclonal, Abeam), anti-NY-ESO-1 (mouse monoclonal, SCBT), anti-Muc16 (mouse monoclonal, SCBT), anti-HSP90 (rat monoclonal, Abeam), anti-TAG72 (mouse monoclonal, Abeam), and anti-HoxA7 (mouse monoclonal, SCBT). Proteins from the ovarian tumor cell lines were solubilized in 50 mM Tris-HC1 (pH7.5), containing 0.3% SDS, 2 mM sodium orthovanadate, 200 mM DTT, 1 mM sodium fluoride, 1 mM sodium pyrophosphate, 1 μg/mL leupeptin, 1 μg/mL aprotinin, 1 μg/mL pepstatin, and 1 mM PMSF on ice. The solubilized proteins were applied to the immunosorbent column and incubated overnight at 4° C. The specific bound proteins released by 0.1M glycine-HC1, pH2.8, neutralized with 1M Tris.

An array assay for tumor antigen-reactive immunoglobulins was performed as follows. Purified exosomal proteins (250 μL containing 20 ng/mL protein) were applied to nitrocellulose membranes using a bio-dot microfiltration apparatus (Bio-Rad Laboratories, Hercules, Calif.). As controls, serially diluted human IgG was spotted onto each membrane as an internal positive control for standardizing blots, diluted mouse and rabbit Ig as a negative control, and peroxidase-conjugated Ig samples as a reagent control and for orientation. Membranes were blocked with 5% BSA and then washed 3 times with TBS plus 0.1% Tween-20 and twice with TBS. Sera (diluted 1:100) from known cancer patients and non-cancer bearing controls were incubated with the membranes overnight at 4° C., incubated with peroxidase-conjugated anti-human IgG, and visualized by ECL. The resulting film was imaged and analyzed using Kodak analysis software. Pixel values for all spots were obtained and comparisons between membranes were performed after standardization to the internal positive control. The cutoff for positive values was set as the maximum standardized pixel value of initial 20 control samples.

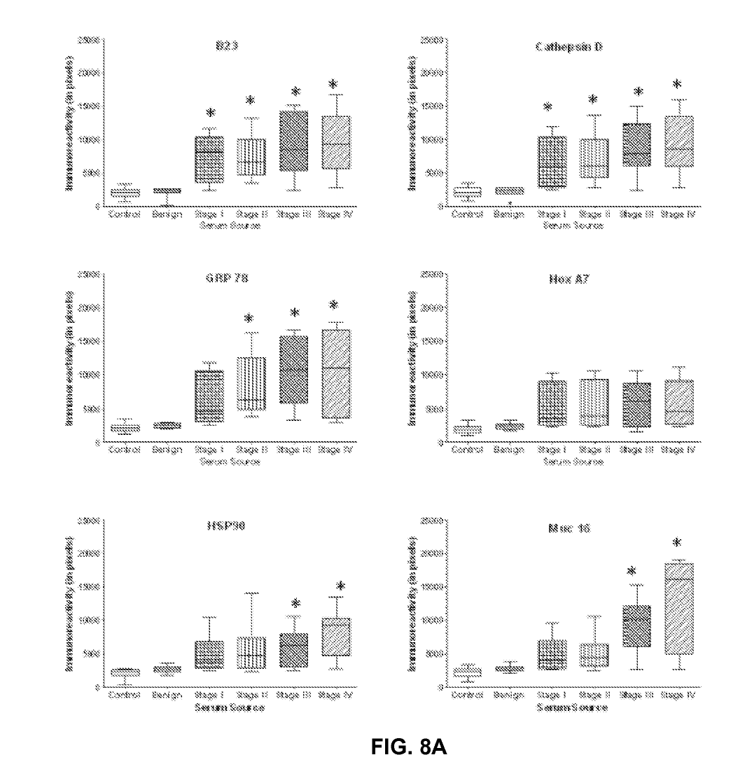

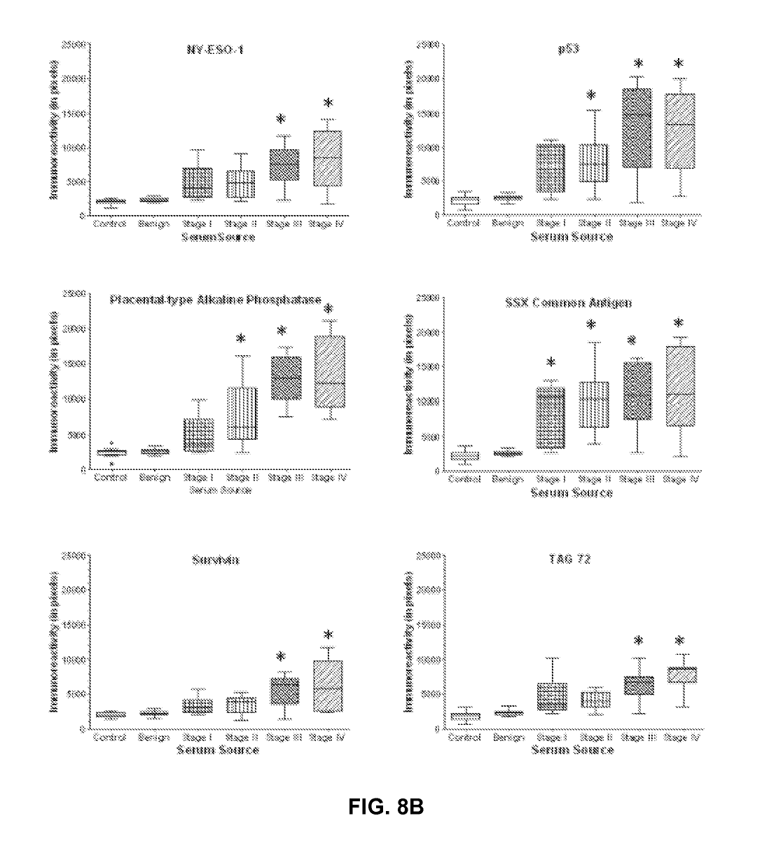

The results show reactivity of some antigens with cancers including pancreatic, lung, breast, and colon cancers, as well as ovarian cancers. Using a dot-blot array to define reactivity, sera from normal female controls, women with benign ovarian disease and ovarian cancer patient, the mean pixels of each antigen were determined and plotted (FIG. 8A-B). The immunoreactivities for both normal controls and women with benign disease were considered negative to all antigens tested. The means for all cancer groups were statistically different from control and benign cases. For most antigens tested, while the mean reactivity (pixel values) was greater in Stages III and IV disease than in early stage disease (Stages I and II), the differences were not significant. However, for Muc16, p53, PLAP and survivin, the reactivities were significantly greater in advanced than early stage disease.

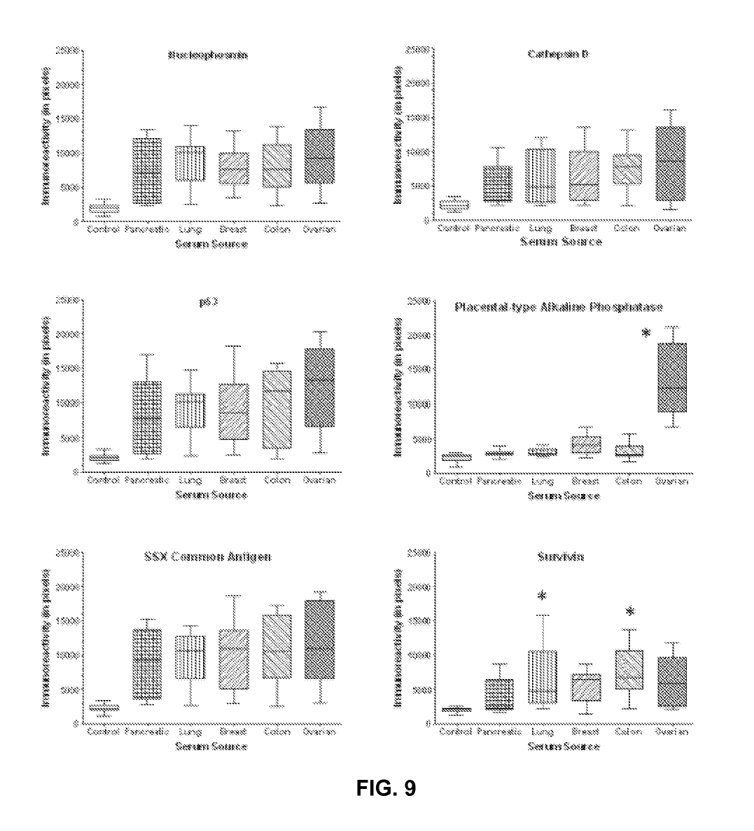

Repeating the study with sera from women with advanced pancreatic, lung, breast, and colon cancers indicated that all cancer patients tested generated autoantibodies recognizing nucleophosmin, cathepsin D, p53, and SSX antigens, compare with female controls and ovarian cancer patients (FIG. 9). Only ovarian cancer patients appear to recognize placental type alkaline phosphatase (PLAP). Patients with lung and colon cancer appear to more strongly recognize survivin.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-14. (canceled)

15. A method of staging ovarian cancer in a subject, the method comprising:
obtaining a sample comprising IgG-type antibodies from the patient;
contacting the sample with ovarian tumor cell derived antigens, under conditions sufficient for the formation of antibody-antigen complexes;
determining the subclasses of the IgG antibodies bound to the antigens in the complexes;
determining the relative immunoreactivity of the subclasses; and
identifying the subject as having stage I, stage II or stage III ovarian cancer based on the relative immunoreactivity of the subclasses.

16. The method of claim 15, wherein the subclasses are IgG1, IgG2, IgG3, and IgG4.

17. The method of claim 16, wherein the presence of relative immunoreactivity of IgG2 >IgG3 indicates a diagnosis of stage I ovarian or stage II ovarian cancer; and the presence of relative immunoreactivity of IgG2≧IgG3 indicates stage III ovarian cancer.

18. The method of claim 17, wherein the presence of relative immunoreactivity of IgG2>IgG3>IgG1=IgG4 indicates a diagnosis of stage I ovarian cancer, the presence of relative immunoreactivity of IgG2>IgG3>IgG1>IgG4 indicates a diagnosis of stage II ovarian cancer, and the presence of relative immunoreactivity of IgG2≧IgG3=IgG4>IgG1 indicates stage III ovarian cancer.

19. The method of claim 16, wherein the ovarian tumor cell-associated antigens are isolated from cytosol of ovarian tumor cells, isolated from the membrane of ovarian tumor cells, or isolated from nuclei of ovarian tumor cells, and the method further includes comparing formation of antigen-antibody complexes with the antigens isolated from the cytosol, nuclei, and membrane of ovarian tumor cells, wherein the relative immunoreactivity of at least one subclass selected from IgG1, IgG2, IgG3, and IgG4 with antigens isolated from the cytosol, nuclei, or membrane of ovarian tumor cells indicates whether the subject has stage I, stage II, or stage III ovarian cancer.

20. The method of claim 19, wherein the stage of ovarian cancer is determined according to the following table:

| Stage | IgG | Reactivity |
|---|---|---|
| I | IgG2, IgG3 | Nuclear > Membrane >> |
| | | Cytosol |
| | IgG1, IgG4 | Membrane > Cytosol >> |
| | | Nuclear |
| II | IgG2, IgG3 | Nuclear > Membrane > |
| | | Cytosol |
| | IgG1, IgG4 | Nuclear > Membrane >> |
| | | Cytosol |
| III | IgG2, IgG3 | Nuclear ≧ Membrane > |
| | | Cytosol |
| | IgG1, IgG4 | Nuclear ≧ Membrane > |
| | | Cytosol |

21. The method of claim 19, wherein:
the presence of primary immunoreactivity of IgG2 with antigens isolated from the nuclei of ovarian tumor cells of greater than 40 kD indicates stage I or II ovarian cancer; and
the presence of primary immunoreactivity of IgG2 with antigens isolated from the membrane of ovarian tumor cells of less than 40 kD indicates stage III ovarian cancer.

22. The method of claim 15, wherein the ovarian cancer cell-derived antigens are isolated from UL-B, UL-O, or UL-6 cell lines.

23. The method of claim 15, wherein the sample comprises serum.

24. The method of claim 15, wherein the subject is known to have or is suspected of having ovarian cancer.

25. The method of claim 15, wherein the subject is human.

26. The method of claim 15, further comprising administering a cancer treatment to the subject.

27. A method of diagnosing the presence of ovarian cancer in a subject, the method comprising:
obtaining a sample comprising antibodies from the patient;
contacting the antibodies in the test sample with ovarian tumor cell-derived antigens, wherein the ovarian tumor cell-derived antigens are isolated from nuclei of ovarian tumor cells, under conditions sufficient for the formation of antibody-antigen complexes;
detecting formation of antigen-antibody complexes between IgG2 and the nuclear antigens isolated from ovarian tumor cells; and
identifying a subject having IgG2 reactive with nuclear antigens isolated from ovarian tumor cells as having ovarian cancer.

28. The method of claim 27, further comprising comparing a level of formation of antigen-antibody complexes between IgG2 present in the sample from the subject and nuclear antigens isolated from ovarian tumor cells with a level of formation of antigen-antibody complexes in a control sample comprising nuclear antigens isolated from ovarian tumor cells and IgG2 present in a sample from a control subject who does not have cancer; and
identifying a subject as having ovarian cancer based on the presence of an elevated level of formation of antigen-antibody complexes between IgG2 in the sample from the subject and the nuclear antigens as compared to the level of antigen-antibody complexes in the control sample.

29. The method of claim 27, wherein the sample comprises serum.

30. The method of claim 27, wherein the subject is human.

31. A method of detecting ovarian cancer in a subject, the method comprising:
obtaining a sample comprising antibodies from the subject;
contacting the sample with one or more tumor-associated antigens selected from the group consisting of: heterogeneous nuclear ribonucleoprotein (HNRNP A2/B1), non-metastatic cells 1/non-metastatic cells 2 (NME1/NME2), zinc finger DHHC-type containing 7 isoform 2, pyridoxal kinase, and peroxiredoxin, under conditions sufficient for the formation of antibody-antigen complexes;
detecting the formation of antibody-antigen complexes, thereby detecting the presence of autoantibodies to the one or more tumor-associated antigens;
diagnosing the subject with ovarian cancer based on the presence of autoantibodies to the one or more tumor-associated antigens in the sample; and.
administering a treatment for ovarian cancer to the identified subject.

32. The method of claim 31, wherein the antibodies are IgG2 or IgG3 subclass.

33. The method of claim 31, wherein the subject is human.

34. A method of detecting the presence of Stage I ovarian cancer in a subject, the method comprising:
obtaining a sample comprising antibodies from the subject;
contacting the sample with one or more tumor-associated antigens selected from the group consisting of: heterogeneous nuclear ribonucleoprotein (HNRNP A2/B1), non-metastatic cells 1/non-metastatic cells 2 (NME1/NME2), and pyridoxal kinase, under conditions sufficient for the formation of antibody-antigen complexes;
detecting the formation of antibody-antigen complexes, thereby detecting the presence of autoantibodies to the one or more tumor-associated antigens;
identifying the subject as having stage I ovarian cancer based on the presence of autoantibodies that bind specifically to HNRNP A2/B1, NME1/NME2, or pyridoxal kinase; and
administering a treatment for ovarian cancer to the identified subject.

35. The method of claim 34, wherein the subject is human.

36. The method of claim 34, wherein the antibodies are IgG2 or IgG3 subclass.

37. A method of detecting the presence of Stage II or III ovarian cancer in a subject, the method comprising:
obtaining a sample comprising antibodies from the subject;
contacting the sample with one or more tumor-associated antigens selected from the group consisting of: zinc finger DHHC-type containing 7 isoform 2 and peroxiredoxin, under conditions sufficient for the formation of antibody-antigen complexes; and
detecting the formation of antibody-antigen complexes, thereby detecting the presence of autoantibodies to the one or more tumor-associated antigens;
identifying the subject as having stage II or III ovarian cancer based on the presence of autoantibodies that bind specifically to zinc finger DHHC-type containing 7 isoform 2 or peroxiredoxin; and
administering a treatment for ovarian cancer to the identified subject.

38. The method of claim 37, wherein the subject is human.

39. The method of claim 37, wherein the sample comprises serum.

40. The method of claim 37, wherein the antibodies are IgG2 or IgG3 subclass.
